(19) United States
(12) Patent Application Publication
(10) Pub. No.: US 2005/0182037 A1
(43) Pub. Date: Aug. 18, 2005

(54) Composition for the regulation of the human immune system and the prevention and treatment of diseases thereof

(76) Inventors: Alan Fergusson, Alberta (CA); John Charles Davidson, Alberta (CA)

Correspondence Address: ADE & COMPANY, 1700-360 Main Street, Winnipeg Manitoba, R3C 3Z3, CA

(21) Appl. No.: 11/106,446

(22) Filed: Apr. 15, 2005

Related U.S. Application Data
(63) Continuation-in-part of application No. PCT/CA03/01451, filed on Sep. 24, 2003.

(30) Foreign Application Priority Data
Oct. 16, 2002 (CA) 2,407,429

(51) Int. Cl. A61K 31/56; A61K 31/202

(52) U.S. Cl. 514/171; 514/560

(57) ABSTRACT

A nutritional supplement composed of phytosterols, anti-oxidants, and other complexes, including hydrolysates of fatty acids, amino acids, peptides, proline rich polypeptides and digestive enzymes is described. The nutritional supplement may be used by individuals suffering from or at risk of developing immune system diseases; breast cancer, colon and prostate cancer; HIV infection; or enlarged prostate. In the preferred form, the invention comprises phytosterols and antioxidants, together with hydrolysates of fatty acids derived from fish, amino acids, short chain peptides, proline rich polypeptides and digestive enzymes, and a microcellulose filler.

### PRIOR APPLICATION INFORMATION

This application is a continuation-in-part of International Application PCT CA03/01451, filed Sep. 24, 2003 which in turn claims the benefit of Canadian Patent Application 2,407,429, filed Oct. 16, 2002.

### FIELD OF THE INVENTION

This invention relates to the field of nutritional supplements.

### SUMMARY OF THE INVENTION

According to a first aspect of the invention, there is provided a single-dose supplement comprising: 300 mg phytosterols; 50 mg essential fatty acid complexes; 20 mg antioxidants; and 30 mg micro-cellulose filler.

According to a second aspect of the invention, there is provided a supplement comprising: 29% to 75% beta-sitosterol; 10% to 50% essential fatty acid complexes; and 5% to 50% antioxidants.

According to a third aspect of the invention, there is provided a supplement comprising: 29% to 75% phytosterols; 10% to 50% essential fatty acid complexes; and 5% to 50% antioxidants.

According to a fourth aspect of the invention, there is provided the use of a composition for treating prostate cancer, said composition comprising as an active ingredient a combination of phytosterols, antioxidants and fatty acid complexes.

According to a fifth aspect of the invention, there is provided the use of a composition for treating allergies, said composition comprising as an active ingredient a combination of phytosterols, antioxidants and fatty acid complexes.

According to a sixth aspect of the invention, there is provided a composition comprising: 1-95% phytosterols; 1-95% essential fatty acid complexes; and 1-95% antioxidants.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, the term “treating” in its various grammatical forms refers to, curing, reversing, attenuating, alleviating, minimizing, suppressing or halting the deleterious effects of a disease state, disease progression, disease causitive agent or other abnormal condition.

Described herein is a new composition or nutritional supplement composed of natural products. In some embodiments, the supplement may be used by individuals desirous of regulating their immune system, for example, to prevent and treat diseases thereof. As discussed below, the composition combines phytosterols with anti-oxidants and fatty acid complexes.

In one embodiment, the composition or supplement comprises: 29% to 75% phytosterols; 10% to 50% essential fatty acid complexes; and 5% to 50% antioxidants. The supplement or composition may be arranged in the form of a capsule or other suitable, ingestible format, such as a tablet, pill or other similar form known in the art.

In a preferred embodiment, the supplement or composition comprises 29% to 75% beta-sitosterol; 10% to 50% essential fatty acid complexes; and 5% to 50% antioxidants.

In a yet further preferred embodiment, there is provided a single-dose supplement or composition comprising: 300 mg phytosterols; 50 mg essential fatty acid complexes; 20 mg antioxidants; and 30 mg micro-cellulose filler. As discussed herein, this composition may be taken once daily or twice daily.

In some embodiments, the above-described compositions are used for treating, preventing or ameliorating symptoms associated with an immune system disease. In these embodiments, the composition or supplement at a dosage, concentration or range described above is administered to an individual in need of such treatment. Examples of immune system diseases include but are by no means limited to colds, flu, diabetes, allergies, asthma, pneumonia, fibromyalgia, HIV infection, hepatitis C infection, and multiple sclerosis. In a preferred embodiment, the composition at dosages, concentrations or ranges described above is used to treat or prevent pneumonia, fibromyalgia or a hepatitis C infection. In a yet preferred embodiment, the composition or supplement described above is used to treat an immune system disease as described above by administering the composition at a concentration or dosage as described to an individual in need of such treatment.

The inherent ingenuity of the invention is the discovery of the range of the amount of beta-sitosterol that is the most effective to achieve the goal of regulating the Human Immune System and the prevention and/or treatment of diseases thereof. As a result, this new composition need only be taken once a day to maintain general health.

The further uniqueness of the invention is the discovery that combining phytosterols with anti-oxidants and fatty acid complexes produces a synergistic effect whereby the cumulative total effect of the composition is greater than the sum of the individual effects of the respective components. As a result, this new composition optimizes the immune system to deal with viral and bacterial infections, while also protecting the body from degeneration due to free radical damage.

Additionally, the invention is original and novel because it can not only assists in preventing possible causes of cancer, but, it inhibits or reduces cancer cell growth in breast, colon and prostate cancer cases, and facilitates the removal of the dead tissue arising from the death of the said tumors. That is, the composition can modulate or optimize, as opposed to only boosting, the immune system, as discussed below.

The utility of said invention is further demonstrated and confirmed by the fact that it accomplishes its goals with none of the physical or psychological side effects of traditional treatments such as radiation, chemotherapy and surgery.

The basis of the present invention is the use of a combination of phytosterols, anti-oxidants and fatty acid complexes for the following purposes:
1. To modulate the Human Immune System;
2. To prevent or treat diseases of the Human Immune System, for example but by no means limited to:
Common cold and flu;
Microorganism infections, for example viral, yeast and bacterial infections;
Allergies;
Rheumatoid Arthritis;
Lupus;
Chronic Fatigue Syndrome;
Fibromyalgia;
Asthma;
Diabetes; and
Eczema.
3. To inhibit or reduce cancer cell growth in breast, colon and prostate cancer cases;
4. To maintain CD4 Lymphocyte counts in HIV patients;
5. To treat enlarged prostate problems; and,
6. As an anti-inflammatory agent.

As discussed above, the compositions described herein act to modulate the immune system. Specifically, the compositions act to restore normal functions in an individual in need thereof. As will be apparent to one of skill in the art, this explains how the compositions are able to treat diseases or conditions characterized by immune activation, such as autoimmune disorders such as arthritis and inflammation, as well as diseases characterized by immune suppression or deficiency, such as acquired immune deficiency syndrome. While not wishing to be bound to a specific theory, the inventors believe that the composition acts to modulate immune function such that an immune system that is acting abnormally, that is, is either depressed or chronically activated, returns to normal functioning, that is, returns to the state of activation found in most individuals who are not currently undergoing a challenge to their immune system.

Although preferred compositions to achieve these ends are listed below, they are by no means to be interpreted as limiting the broad scope of the present invention as various other combinations of phytosterols, anti-oxidants and fatty acid complexes may achieve the same beneficial results.

The preferred composition of the present application consists of 400 mg capsules containing 300 mg of phytosterols (a minimum of which is 117 mg of beta-sitosterol), 50 mg essential fatty acid complexes, 20 mg broad spectrum antioxidants, and 30 mg micro-cellulose filler. As will be appreciated by one of skill in the art, this represents an example of an effective amount of the composition. Other suitable concentrations and dosages may be determined using the ranges described herein. As will be apparent to one of skill in the art, as used herein, “effective amount” refers to an amount that achieves the desired effect.

In another preferred form, the composition comprises from 29% to 75% beta-sitosterol, 10% to 50% essential fatty acid complexes and from 5% to 50% antioxidants. As will be appreciated by one of skill in the art, other suitable, pharmaceutically acceptable fillers may also be used in the invention.

In other embodiments, there is provided a composition comprising: 1-95% phytosterols; 1-95% essential fatty acid complexes; and 1-95% antioxidants. The composition may further include 1-95% filler, for example, micro-cellulose filler. In other embodiments, 5-95% of the phytosterols may be beta-sitosterol.

A further innovative feature of the invention is the discovery that the essential fatty acid complexes together are not only themselves an immune system modulator, but also facilitates the body's absorption of beta-sitosterol and antioxidants.

This invention is a composition of natural products that is designed to stimulate the body to marshal its own natural defenses against allergies and other immune related disorders, breast, colon and prostate cancer, and AIDS, as opposed to alleviating or preventing these disorders with chemical or synthetic products. Many of the traditional treatments have severe side effects, which in the case of prostate cancer can include impotence and incontinence, and in the case of chemotherapy, severe depression of the immune system. Traditional treatments also only treat the tumors, i.e. the symptoms, and not the underlying cause, and in the case of chemotherapy, leave dead tissue to be removed from the body.

One of the suspected causes of cancer is free radical damage on cellular components such as DNA or the cell membranes. Free radicals are the natural by-products of many processes within and among cells. They are also created by exposure to various environmental factors such as tobacco smoke and radiation. Once formed these highly reactive radicals can start a chain of reactions that can result in damage to cell walls, certain cell structures, and the genetic material within the cells such as DNA.

In the preferred form this composition contains one of the world's most advanced anti-oxidant complexes, Enzogenol™, produced by Enzo Nutraceuticals through a patented process that creates an aqueous extract from Pinus Radiata (pine bark), which is a superior source of proanthocyanidins and flavonoids. This technically advanced anti-oxidant complex provides an excellent source of free radical scavengers:

In addition to scavenging free radicals, the Enzogenol™ in this composition can stimulate phagocytosis, an immune system response in which cells ingest and eliminate harmful micro-organisms and foreign heavy metals, and hence facilitates the removal of the by-products of cell death.

Research has shown that the Human Immune System can be stimulated by beta-sitosterol. This composition contains this remarkable phytonutrient which has been shown to be effective in inhibiting cancer cell growth and accelerating cancer cell death in breast, prostate and colon cancer. It can also reduce metastases to lymph nodes.

Research at the University of New York at Buffalo by Awad, Downey and Fink has shown that beta-sitosterol can inhibit breast cancer cell growth by 66% after 3 days and 80% after 5 days. It can also increase the rate of cell death by 600% (Int. J. Mol. Med 2000 May 5(5)). Research by the same group has also shown that beta-sitosterol can inhibit prostate cancer cell growth by 24% and increase cell death by 400% (Nutr Cancer 1998; 32 (1)). Similar research on colon cancer cells showed a reduction in cell growth of 55% (Anti Cancer Res, 1998 Mar.-Apr. 18 (2a)). The exact mechanism behind the inhibition of cancer cell growth and the increase in apoptosis is not yet fully understood, but may be related to the increase in T-cell proliferation produced by beta-sitosterol (Int. Jnl. Immuno Pharmacol 1996 vol 18 Bouic et al). Beta-sitosterol has been shown to maintain CD4 lymphocyte count in AIDS patients (AIDS Bulletin Summer 97).

The potential benefits of the antioxidants together with the beta-sitosterol to scavenge free radicals and to kill and remove tumors can only be realized if the human body actually absorbs both the anti-oxidant complex and the betasitosterol. The role of the essential fatty acid complex is to ensure that the antioxidant complex and the beta-sitosterol are absorbed. The essential fatty acid complex itself is a blend of essential fatty acids, amino acids, proline rich peptides and short chain poly-peptides. This complex also happens to be an immune system modulator, and consequently also supports the body's attack on cancer by promoting and supporting the T-cell proliferation initiated by the beta-sitosterol.

The combined effects of the antioxidant complex, the beta-sitosterol and the essential fatty acid complex on breast, colon and prostate cancers are to reduce the risk of the formation of new cancers through the more effective scavenging of free radicals, to inhibit the growth and increase the rate of cell death of the tumors, and to assist the body in disposing of dead tumor cells.

This formulation has been used to treat a 68 year old male with a PSA of 17. Over a six month period his PSA, (the normal measure of prostate cancer which would normally increase over time), dropped to 11.8, a reduction of over 30%, with no side effects.

The combined immune modulating properties of beta-sitosterol, antioxidant complex, and the essential fatty acid complex also make this formulation an excellent anti-allergen with no side effects which is also new and unique in the field of anti-allergens. This formulation has been used to treat several adults with ages ranging between 22 and 52 with excellent results occurring in the space of five to six days, again with none of the side effects normally associated with anti-allergens.

Early trials of this unique formulation have also shown the capacity to alleviate such immune related disorders as rheumatoid arthritis, again with none of the adverse side effects normally associated with the remedies traditionally used in this area.

In addition, the effectiveness of beta-sitosterol in maintaining the CD4 lymphocyte count in AIDS patients can be increased by the addition of the antioxidant and essential fatty acid complexes in this formulation, again providing an important new application for this new and unique formulation.

Normally capsules are taken once a day, in a preferred embodiment, at least one hour or at least half an hour before and at least 2 hours after food, with water or fruit juice (NOT milk or any other fat containing liquid). As will be appreciated by one of skill the art, these time frames and frequencies are suggested for most users, but other users may find best results taking the capsule more frequently or less frequently, or, for example, with meals or closer to meals.

The term “phytosterols” is to be construed to include, but not to be restricted to, beta-sitosterol, stigmasterol, campesterol, brassicasterol together with their associated glucosides.

The term “antioxidant complex” is to be construed to include, but not to be restricted to, a broad range of proanthocyanidins, flavonoids and polyphenols.

The term “essential fatty acid complex” is to be construed to include, but not restricted to, hydrolysates of fatty acids derived from fish and synergistically incorporated on plant protein carriers, together with amino acids, essential fatty acids, peptides, proline rich poly peptides, and various digestive enzymes.

The following are examples of usages, showing the utility of the composition.

### EXAMPLE I

### Allergies

The composition was used by an individual to treat seasonal allergies. The individual found that one or more of the symptoms associated with allergies, including runny nose, red itchy eyes, aching joints and a feeling of always being tired, were ameliorated following taking the composition. Specifically, users found that the symptoms were ameliorated after 24 hours and remained symptom free after 2 weeks by taking one dosage or capsule per day.

Another individual who suffered from moderate asthma and allergies to dust and animal hair found that taking the composition relieved or ameliorated symptoms including itchy and watery eyes and a runny nose. This individual also reported increased energy and an improved sense of well-being.

Another individual who suffered from allergies to dust mites which led to a chronically stuffed nose that would drip constantly found that these symptoms were ameliorated following taking the composition over an initial regimen and then taking the composition daily on an as needed basis.

Another individual reported allergies to pollen and other seasonal allergies and found that taking the composition over a period of a few weeks ameliorated the allergy-related symptoms, including itchy eyes and sneezing.

### EXAMPLE 2

### Asthma

An individual who suffered from asthma found that following taking the composition regularly for 3 months, the asthma-associated symptoms were ameliorated.

Another individual who suffered from asthma and food allergies and had symptoms including itchy eyes and a runny nose found that within 2 weeks of taking the composition on a regular basis, she was off the antihistamines and her stuffy nose was completely gone. Furthermore, she found that her asthma progressively improved and was able to eat foods that she couldn't eat before without having immediate allergic reactions.

### EXAMPLE 3

### Colds & Flu

One individual who had contracted pneumonia took two capsules of the composition per day over a five day period and found that the symptoms associated with pneumonia were ameliorated.

Another individual who also had a cold develop into pneumonia found that the symptoms associated with the illness were ameliorated during a two month regimen of taking the composition daily.

Another individual took the composition regularly to prevent colds or “flu”. This individual found less frequent occurrences of colds and “flu” and also found that taking the composition on a regular basis provided “an all over feeling of wellbeing”.

### EXAMPLE 4

### Diabetes

A diabetic who was using insulin 3 times per day for the last 12 years, at an average of 50 units a day pf insulin, consisting of 20 units in the morning, 20 units in the afternoon and 10 units before bed found that after having taken the composition daily for 3 weeks that the individual was able to eliminate the night-time units.

### EXAMPLE 5

### Eczema

An individual who suffered from eczema, particularly under her armpits on both sides, would also get eczema on her hands whenever her hands were exposed to any chemicals or detergents (ie doing any cleaning) and even after folding the laundry. She managed the eczema with cortisone cream—a few times per week. After 2 months of taking the composition along with eliminating certain food allergies, her eczema was 90% gone (in places where it had been chronic for years) and her hands (which used to be effected almost constantly) were completely clear.

### EXAMPLE 6

### Fibromyalgia

An individual who suffered from fibromyalgia found that taking one dose of the composition one hour before breakfast helped ameliorate symptoms including pain and insomnia.

Another individual with fibromyalgia reported wakening 34 times per night with severe cramps in their feet and calves. Following taking the composition on a regular dosage regimen, this individual found amelioration of many of the symptoms associated with the fibromyalgia.

### EXAMPLE 7

### Hepatitis C

An individual who suffers from hepatitis C reported a progressive increase in energy during the first 2 weeks of taking the composition.

### EXAMPLE 8

### HIV

An individual having an HIV infection reported that their CD4 level climbed by 300 points following a 3 month regimen of taking the composition.

### EXAMPLE 9

### Multiple Sclerosis

An individual with multiple sclerosis reported increased energy and improved balance on taking the composition.

### EXAMPLE 10

### Urinary Flow

An individual suffering from prostate enlargement reported that following taking the composition, the time between urinations was increased and also reported increased flow.

### EXAMPLE 11

### Pelvic Inflammatory Disease

Several users have reported that the composition as described above alleviated one or more of the symptoms associated with pelvic inflammatory disease.

### EXAMPLE 12

### Rheumatoid Arthritis

An individual with rheumatoid arthritis reported that following taking two capsules of the composition per day for two weeks, many of the symptoms associated with the rheumatoid arthritis were ameliorated.

### REFERENCES

International Journal of Immuno Pharmacology 1996 Vol 18 Bouic et al. Beta-sitosterol and beta-sitosterol glucoside, stimulate human peripheral blood lymphocyte proliferation; implications for their use as an immunomodulatory vitamin combination.
International Journal Molecular Medicine 2000 Vol 5 Awad et al. Inhibition of growth, and stimulation of apoptosis, by beta-sitosterol treatment of human breast cancer cells.
Anti Cancer Research 1998 Vol 18 (1 a) Awad, Holtz et al. Beta-sitosterol inhibits growth of HT-29 human colon cancer cells.
Nutrition and Cancer 1998 Vol 32 (1) von Holtz, Fink et al. Beta-sitosterol induces apoptosis in LNCaP human prostate cancer cells.
AIDS Bulletin 1997 Vol 6 Bouic. Immodulation in HIV/AIDS: The Tygerberg/Stellenbosch University Experience.
South African Journal of Science, 1997 Vol 93 Pegel. The Importance of Sitosterol and Sitosterolin in human and animal nutrition.
British Journal of Urology 1997 Vol 80 Klippel et al. A multi centric, placebo controlled, double blind clinical trial of betasitosterol for the treatment of benign prostatic hyperplasia.
The Lancet 1995 Vol 345 Berges et al. Randomized placebo controlled double blind clinical trial of beta-sitosterol in patients with benign prostatic hyperplasia.
The Arthritis Trust of America Summer 98 Bouic. Sterols/Sterolins the natural, none toxic immuno-modulators and their role in the control of rheumatoid arthritis.
Nutrition Reviews 1992 Vol 50 NO. 7 Block. The data support a role for antioxidants in reducing cancer risks.
Methods in Enzymology 1990 Vol 186 Bors, Heller et al. Flavanoids as anti-oxidants: determination of radical scavenging efficiencies.
The Lancet 1996 Vol 344 Cerruti. Oxi-radicals and cancer.
Biochemical Pharmacology 1990 Vol 39 No. 11 DeWhalley, Rankin et al. Flavanoids inhibit the oxidative modification of low density lipoproteins by macrophages.
The Lancet 1994 Vol 344 Grishan. Oxidants and free radicals in inflammatory bowel disease.
Phytochemistry 1987 Vol 26 No. 9 Husain, Cillard et al. Hydroxyl radical scavenging activity of flavanoids.
The Lancet 1994 Vol 344, Jenner. Oxidative damage in neuerodagenerative disease.
Free Radicals in Diagnostic Medicine, D. Armstrong, Plenum Press New York. Free radical scavenging and antioxidant activity of plant flavanoids.
New Zealand Journal of Science 1973 Vol 16 Markam and Porter. Extractives of pinus radiate bark-phenolic components.
New Zealand Journal of Science 1974 Vol.17 Porter. Extractives of pinus radiate bark-procyanidin constituents.
Free radical Research 1995 Vol 22 No. 4 Rice-Evens, Miller et al. The relative antioxidant activities of plant derived polyphenolic flavanoids.
Biochemical Pharmacology 1998 Vol 37 No 5 Rbak, Gryglewski. Flavanoids are scavengers of super oxide anions.
Free Radical Biology in Medicine 1990 Vol 9 Yuting, Rongliang et al. Flavanoids as super oxide scavengers and antioxidants.

While the preferred embodiments of the invention have been described above, it will be recognized and understood that various modifications may be made therein, and the appended claims are intended to cover all such modifications which may fall within the spirit and scope of the invention.

1. A method of treating or preventing an immune system disease comprising administering to an individual in need of such treatment an effective amount of a composition comprising:
29% to 75% phytosterols;
10% to 50% essential fatty acid complexes; and
5% to 50% antioxidants.

2. The method according to claim 1 wherein the composition comprises 29% to 75% beta-sitosterol; 10% to 50% essential fatty acid complexes; and 5% to 50% antioxidants.

3. The method according to claim 1 wherein the composition comprises 300 mg phytosterols; 50 mg essential fatty acid complexes; 20 mg antioxidants; and 30 mg micro-cellulose filler. As discussed herein, this composition may be taken once daily or twice daily.

4. The method according to claim 1 wherein the immune system disease is selected from the group consisting of colds, flu, diabetes, allergies, asthma, pneumonia, fibromyalgia, HIV infection, hepatitis C infection, urinary flow, pelvic inflammatory disease and multiple sclerosis.

5. The method according to claim 1 wherein the immune system disease is selected from the group consisting of pneumonia, fibromyalgia or a hepatitis C infection.
